(19) United States
(12) Patent Application Publication
(10) Pub. No.: US 2026/0174417 A1
(43) Pub. Date: Jun. 25, 2026

(54) ENDOSCOPIC ULTRASOUND GUIDED ACCESS NEEDLE

(71) Applicant: BOSTON SCIENTIFIC SCIMED, INC., Maple Grove, MN (US)

(72) Inventors: John Thomas FAVREAU, Charlton, MA (US); Perry THOMSON, Somerville, MA (US)

(21) Appl. No.: 19/392,949

(22) Filed: Nov. 18, 2025

Related U.S. Application Data
(60) Provisional application No. 63/736,265, filed on Dec. 19, 2024.

(51) Int. Cl. A61B 10/04; A61B 1/00; A61B 1/005; A61B 10/02; A61B 90/00

(52) U.S. Cl. CPC A61B 10/04; A61B 1/00097; A61B 1/00098; A61B 1/00128; A61B 1/005; A61B 10/0283; A61B 90/37; A61B 2010/045; A61B 2090/3784; A61B 2560/0462

(57) ABSTRACT

A flexible endoscope includes a flexible shaft and an ultrasound transducer. The shaft extends from a proximal end connected to a handle to a distal portion configured to be inserted into a living body to a site adjacent to target tissue to be treated. The shaft includes adjacent to a distal end thereof a port from which a tissue treatment device may be extended out of the shaft along a path. The transducer is within a housing coupled to a portion of the shaft extending distally of the port. The transducer is aimed so that the path passes through a field of view provided by the transducer to a user of the device. The transducer is movable relative to the port to broaden the field of view.

### PRIORITY CLAIM

The present disclosure claims priority to U.S. Provisional Patent Application Ser. No. 63/736,265 filed Dec. 19, 2024; the disclosure of which is incorporated herewith by reference.

### FIELD

The present disclosure relates to an endoscopic ultrasound (EUS) scope.

### BACKGROUND

EUS scopes are used, for example, in procedures for placing a hollow needle to access target anatomy, e.g., an intestinal lumen, and to introduce a guidewire through the needle lumen into the target anatomy. This may be done, e.g., to guide a stenting procedure. Current EUS scopes generally have an optical vision system and an ultrasonic transducer at a distal end of the scope. The optical vision system is often at the distal end of the scope facing distally to provide a view to the user of the environment in front of the scope (e.g., as it is advanced to a target location in the body). As many of these procedures require the deflection of the needle away from the longitudinal axis of the scope, the optical system is often unable to provide a view of the target tissue or of the needle.

The target tissue may be obscured by intervening layers of tissue and so may not be viewable using the optical system. The ultrasound transducer provides a view through intervening tissue but this view is limited to a single plane in which the ultrasound energy is propagated. To obtain a more complete view of the area the user must move the entire scope, which is cumbersome and, during certain parts of the procedure, may be unavailable as this will also move the needle perhaps in an undesirable manner. In addition, if the needle extends out of the plane of the view of the ultrasound transducer (e.g., if the needle bends for any reason) the needle may not be visible to the user.

### SUMMARY

The present disclosure relates to a flexible endoscope. The endoscope includes a flexible shaft extending from a proximal end connected to a handle to a distal portion configured to be inserted into a living body to a site adjacent to target tissue to be treated, the shaft including adjacent to a distal end thereof a port from which a tissue treatment device may be extended out of the shaft along a path; and an ultrasound transducer within a housing coupled to a portion of the shaft extending distally of the port, the transducer being aimed so that the path passes through a field of view provided by the transducer to a user of the device, the transducer being movable relative to the port to broaden the field of view.

In an embodiment, the transducer is movable laterally within the housing with respect to a longitudinal axis of the housing.

In an embodiment, the transducer is rotatable within the housing about to a longitudinal axis of the housing.

In an embodiment, the transducer is rotatable within the housing about an axis perpendicular to a longitudinal axis of the housing.

In an embodiment, the transducer is coupled to a proximal actuator via a rotatable drive shaft and wherein the rotatable drive shaft is coupled to the transducer via a rack and pinion mechanism.

In an embodiment, the port is configured as a lateral port aimed so that the device extended out of the port is aimed away from a longitudinal axis of the housing.

In an embodiment, the lateral port is configured to aim a needle for fine needle aspiration along a path extending laterally away from the flexible endoscope.

In an embodiment, the path is centered within the field of view of the transducer.

In an embodiment, the housing is configured so that, when the transducer is moved relative to the port, the port remains substantially stationary.

In an embodiment, the endoscope further includes a sensor in the housing configured to provide to a control unit data corresponding to a current position so that images of the field of view may be accurately rendered.

In an embodiment, the control unit produces a 3-dimensional map of the field of view.

In an embodiment, the rotatable drive shaft is one of a solid wire, a tube, a coil, and a braid of medical grade metal.

In an embodiment, the medical grade metal is one of nitinol and stainless steel.

In an embodiment, the shaft is covered in a coating of one of PTFE and silicone lubricant.

In addition, the present disclosure relates to a insertion device which includes a handle; a flexible shaft extending along a longitudinal axis distally from the handle to a distal portion configured to be inserted into a living body to a site adjacent to target tissue to be treated, the distal portion of the shaft including a port in a lateral surface thereof from which a tissue treatment device may be extended out of the shaft along a path extending away from the longitudinal axis; and a housing extending distally beyond the port, the housing including an ultrasound transducer therein, the transducer being aimed so that the path passes through a field of view provided by the transducer to a user of the device, the transducer being movable within the housing to broaden the field of view.

In addition, the present disclosure relates to a method for treating tissue which includes inserting into a living body a flexible endoscope, the endoscope including a flexible shaft which extends from a proximal end connected to a handle to a distal portion configured to be inserted into the living body to a site adjacent to target tissue to be treated; extending out of the distal portion a tissue treatment device via a port adjacent to a distal end of the shaft; and moving within a housing of the endoscope distal to the port, an ultrasound transducer of the endoscope so that a field of view generated by the transducer includes a path along which the device extends.

In an embodiment, the transducer is movable laterally within the housing with respect to a longitudinal axis of the housing.

In an embodiment, the transducer is rotatable within the housing about to a longitudinal axis of the housing.

In an embodiment, the transducer is rotatable within the housing about an axis perpendicular to a longitudinal axis of the housing.

In an embodiment, the transducer is coupled to a proximal actuator via a rotatable drive shaft and wherein the rotatable drive shaft is coupled to the transducer via a rack and pinion mechanism.

### BRIEF DESCRIPTION OF DRAWINGS

FIG. 1 shows a perspective view of a handle and a proximal portion of an EUS scope for use in an EUS fine needle aspiration procedure according to an embodiment;

FIG. 2 shows a distal portion of an EUS scope according to an embodiment for use in conjunction with the handle and proximal portion of FIG. 1;

FIG. 3a shows a perspective view of the viewing area of the distal portion of the EUS scope of FIG. 2;

FIG. 3b shows a perspective view of the viewing area provided by a standard EUS scope;

FIG. 4 shows a perspective view of a distal end of an EUS scope according to a further embodiment;

FIG. 5 shows a perspective view of the viewing area of the distal portion of the EUS scope of FIG. 4.

FIG. 6 shows a partially cross-sectional perspective view of the distal portion of the EUS scope of FIG. 4;

FIG. 7 shows a perspective view of the distal end of an EUS scope according to a still further embodiment;

FIG. 8 shows a partially cross-sectional end view of the distal end of the EUS scope of FIG. 7; and

FIG. 9 shows a perspective view of the distal end of the EUS scope of FIG. 6.

### DETAILED DESCRIPTION

The present disclosure may be further understood with reference to the following description and the appended drawings, wherein like elements are referred to with the same reference numerals. The exemplary embodiments describe scope devices having ultrasound systems for providing an enhanced view of target tissue as well as devices to be used in conjunction with the scopes. Although the exemplary embodiments describe ultrasound guided fine needle aspiration procedures, those skilled in the art will understand that various other devices may be used in conjunction with such scopes without departing from the scope of this disclosure. As used in this application the terms distal and proximal connote a direction away (distal) and toward (proximal) a user of the device. Thus, the handle of the device is described as the proximal end while the distal end is the portion of the device configured to be inserted into the body to a location proximate to target tissue to be treated.

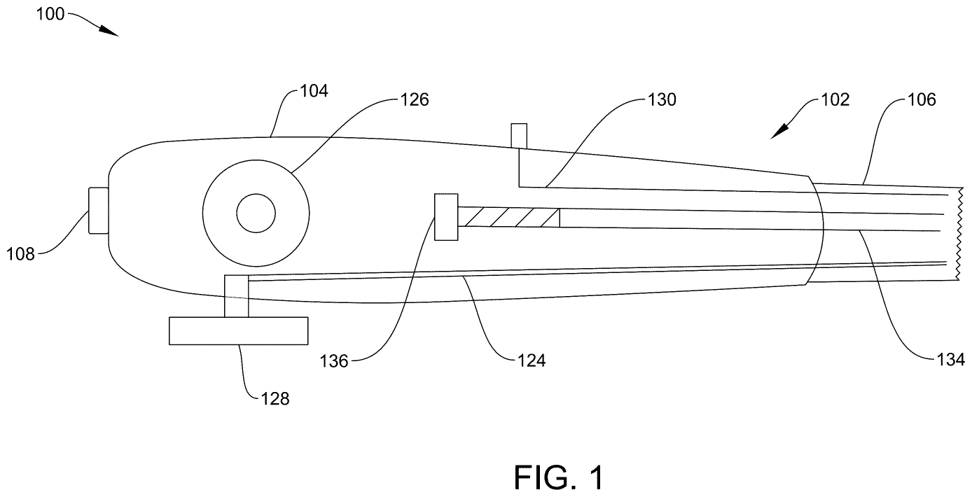

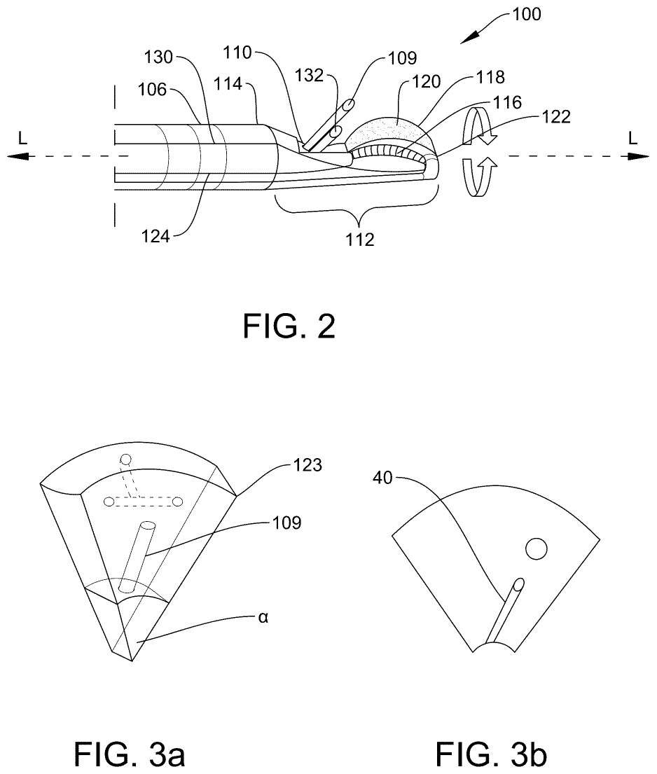

FIGS. 1 and 2 show an endoscopic ultrasound system 100 according to an embodiment. The system 100 includes an endoscopic device 102 including a handle 104 and a flexible shaft 106 extending distally therefrom. As would be understood by those skilled in the art, the handle 104 which is exemplary only, includes a port 108 for the introduction of devices (e.g., a needle 109 configured for endoscopic fine needle aspiration) to target sites within the body via a working channel extending through the shaft 106 to a distal opening such as a lateral needle port 110 shown in FIG. 2.

The system 100 includes a distal portion 112 extending distally from the distal end 114 of the shaft 106 which, in use, is positioned adjacent to target tissue to be visualized and/or treated as would be understood by those skilled in the art. The distal portion 112 includes an ultrasound transducer 116 movably mounted within a housing 118 of the distal portion 112. As those skilled in the art will understand, the housing 118 includes a window 120 through which the ultrasound energy is transmitted to fluids surrounding the distal portion 112 so that energy generated by the transducer 116 can propagate into the tissue to be visualized. The transducer 116 of this embodiment is movably received within the housing 118 and coupled to a mechanism (described below) permitting a user to rotate the transducer 116 within the housing 118 to widen the field of view provided to the user by the transducer 116.

Specifically, the transducer 116 is mounted within the housing 118 so that the transducer 116 may rotate about a longitudinal axis L of the shaft 106 through a predetermined angular range α so that the ultrasound energy can provide to the user of the system 100 a field of view 123 as shown in FIG. 3a that is wider than the planar field of view provided by standard ultrasound scope devices as shown in FIG. 3b. Those skilled in the art will understand that the term “axis” as used in this application to denote the axis L meets the geometric definition of an axis only when the shaft 106 is straight. However, when the shaft 106 extends along a curved path, the term longitudinal axis L refers to the path connecting the center points of the shaft 106 along its length.

As would be understood by those skilled in the art, standard ultrasound endoscopes generally provide a field of view 42 which encompasses a part of a single plane projecting out of the housing within which the transducer is seated and this plane is generally selected to align with a desired line of sight (e.g., including a plane within which a needle extended out of a distal portion of the endoscopic device generally occupies). For example, for an ultrasound endoscope having a side port out of which a needle 40 is extended a planar field of view 42 is generally provided by the endoscope. Those skilled in the art will understand that this field of view 42 may be widened by moving and/or rotating the endoscope but that this may be difficult or impossible at certain times during a procedure (e.g., where any device has been extended out of the port or has even been inserted into tissue). Thus, it is difficult and, at times, impossible for a user to visualize using the ultrasound tissues and/or devices that are not currently in the plane of the field of view 42.

The present embodiments permit a user to move and/or rotate the transducer 116 while holding the needle or other device extended out of the scope stationary to broaden the field of view 123 of the device 102 so that the user may visualize adjacent tissue structures, devices extended from the device 102 outside the plane of the field of view 42, etc. In addition, by tracking the position of the transducer 116, the system 100 may use the information provided via the transducer 116 to generate 3 dimensional maps of tissue structures in a known manner. Specifically, by tracking the position and orientation of the transducer 116 within the housing 118 (e.g., while holding the position of the distal portion 112 stationary), the system may combine scans of different planes of tissue to generate the 3-dimensional map.

As indicated above, the transducer 116 is mounted within the housing 118 so that the transducer 116 may be rotated about the axis L through an angular range α which in this embodiment is between 15 and 180 degrees. The transducer 116 of this embodiment is coupled to a longitudinally flexible, torsionally stiff shaft 124 that extends through the shaft 106 to the handle 104. As would be understood by those skilled in the art, the shaft 106 could be composed of a solid wire, a tube, a coil, a braid, or a multi-layered shaft containing multiple of these constructions. The primary material of the shaft 106 may be, for example, any medical grade metal such as nitinol stainless steel or similar alloys. Furthermore, if desired, the shaft 106 may also be covered in a lubricious coating such as PTFE or a wipe on lubricant such as a silicone lubricant. The handle 104 includes a first actuator 126 that is used in a conventional manner to steer the device 102 (e.g., by bending the distal portion 112 in a desired manner) and a second actuator 128 that is coupled to the shaft 124 so that rotation of the second actuator 128 rotates the shaft 124 and, consequently, the transducer 116 within the housing 118.

As would be understood by those skilled in the art, generally when a long thin shaft such as the shaft 124 is rotated, the amount of rotation applied at an actuator (e.g., the second actuator 128) is not transmitted one to one along the length of the shaft 124. That is, a certain amount of “wind-up” will generally occur along the length of the shaft 124 so that rotation of the proximal end of the shaft 124 through an angle (e.g., 30 degrees) will result in rotation of the distal end of the shaft 124 by a lesser amount (e.g., 25 degrees). As would be understood by those skilled in the art, the system 100 must account for this “wind-up” in order to determine accurately the position/orientation of the transducer 116 if a 3-dimensional map is to be accurately constructed. For that purpose, in this embodiment, a sensor 122 is mounted with the housing 118 to detect the actual angular position of the transducer 116 within the housing 118. Those skilled in the art will understand that the sensor 122 may be, for example, a miniaturized magnetic or optical rotary encoder.

Depending on the encoder, there may be a need to calibrate the position of the shaft prior to use as would be understood by those skilled in the art. The sensor 122 is coupled to the proximal portion (e.g., the handle 104) via a conductor 130 so that this information is transmitted to a control device (e.g., a processor or a computer (not shown)) that receives information from the transducer 116 to construct the 3-dimensional map. As indicated in FIG. 3a, this generates a field of view 123 formed as a portion of a cylinder having a circumferential range about the axis L equal to the range α through which the user rotates the transducer 116.

The system 100 of this embodiment and the other embodiments herein are described as configured to perform Endoscopic Fine Needle Aspiration (EUS). However, those skilled in the art will understand that this is exemplary only and that embodiments may be adapted to any endoscopic procedure in which a wider field of view is desired under circumstances where movement of the distal end of the endoscope in the manner required to obtain this field of view with known scopes is not possible or desirable. The device 102 of the system 100 of this embodiment includes the port 110 formed in the distal portion 112 proximally of the transducer 116 on a lateral side of the housing 118 so that a needle 109 advanced distally out of the port 110 will extend away from the axis L into the field of view 123.

To facilitate the aiming of the needle 109 as desired by the user the device 102 includes an elevator 132 adjacent to the port 110 so that movement of the elevator 132 relative to the port 110 can adjust (increase or decrease) an angle of the needle 109 relative to the axis L (in a plane including the axis L) as the needle 109 extends out of the port 110. The elevator 132 is coupled to the handle 104 and a third actuator 136 so that a user may adjust the position of the elevator 132 as would be understood by those skilled in the art. Thus, a user may rotate the transducer 116 through a desired range α to observe tissue structures at and surrounding a target tissue site and may continue to visualize the needle 109 (without moving the distal portion 112) even if the needle 109 is deflected out of a target plane (e.g., bent so that it does not reside within a plane including the axis L).

In use, the user advances the device 102 into the body (e.g., along a natural body lumen such as the alimentary canal) until the distal portion 112 is positioned adjacent to a target tissue site to be analyzed and/or treated. As would be understood by those skilled in the art, this may be done in a conventional manner using, for example, an optical vision system (not shown) including a lens on a distal end of the distal portion 112 facing out along the axis L. Once the user has positioned the distal portion 112 as desired relative to the target tissue site, the user may rotate the distal portion 112 until the transducer 116 is oriented as desired relative to the target tissue site. For example, this may be a position in which the window 120 faces the target tissue site with the transducer 116 aimed at a center of the target tissue site when in the middle of the angular range α.

The user may then operate the second actuator 128 to rotate the transducer 116 through a desired angular range α0 where the range α may be selected by a user based on a size and shape of the target tissue site, a desired margin around the target tissue site, key anatomical structures adjacent to the target tissue site, etc. limited of course by a maximum value α_{MAX} (e.g., 15-180 degrees) of the range α. Then, using information on the actual angular position of the transducer 116 relative to the housing 118, the processor/computer can generate a 3-dimensional map of the target tissue site and surrounding tissue within the field of view 123 in a known manner.

The user may then insert a needle 109 through the port 108 and advance the needle 109 distally through the working channel of the device 102 until the distal end of the needle 109 extends distally out of the port 110. Depending on the location of the target tissue site (e.g., a site at which the needle 109 is to penetrate tissue of the wall of the lumen within which the device 102 is located), the user can manipulate the third actuator 136 to increase or decrease the angle of the needle 109 as it is advanced distally out of the port 110. At this same time, the user (or any other person) may operate the second actuator 128 to visualize the needle and the target tissue site through any desired angular range α. Those skilled in the art will understand that the device 102 may also include an optional servo motor for automatically sweeping the transducer 116 through a desired range α during all or any part of a procedure as Automation of this would allow the user to focus on performing their procedure without manually controlling operation of the transducer.

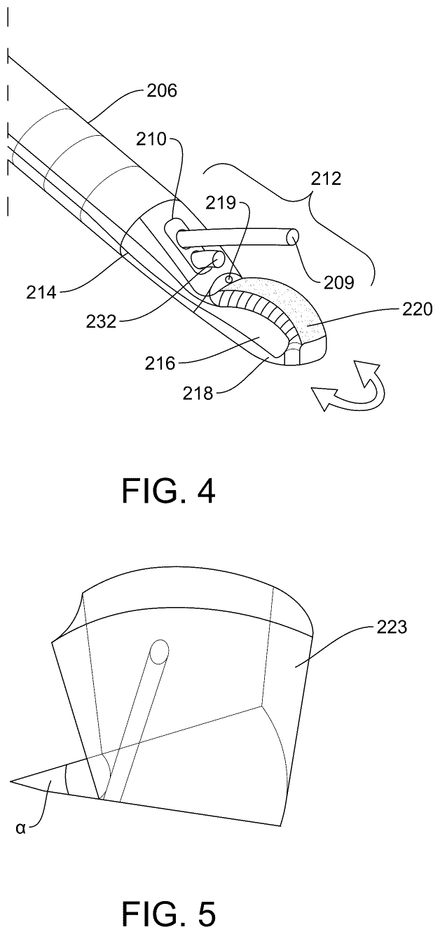

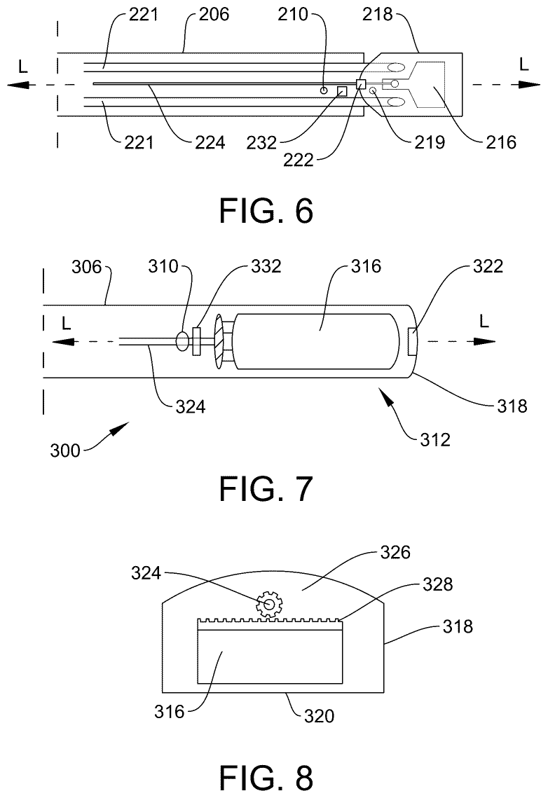

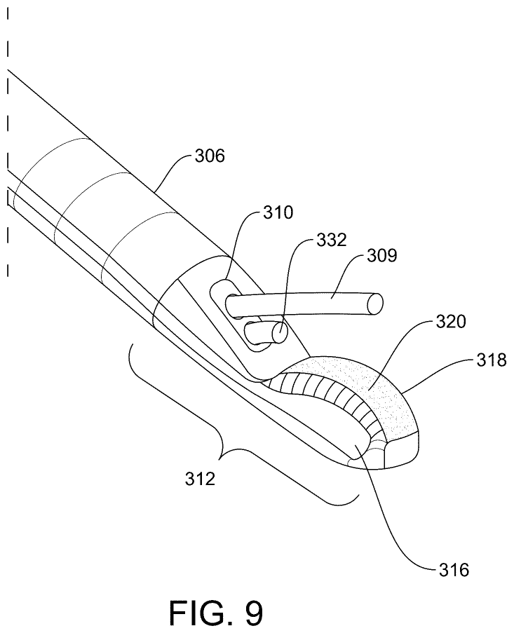

FIGS. 4-6 show a distal portion 212 of a device 202 according to a further embodiment that is constructed substantially similarly to the device 102 described above except as indicated specifically below. Specifically, the device 202 is substantially the same as the device 102 except for the mechanisms and actuators for moving the transducer of this embodiment. The distal portion 212 extends distally from the distal end 214 of the shaft 206. The distal portion 212 includes an ultrasound transducer 216 mounted within a housing 218 of the distal portion 212. The housing 218 includes a window 220 through which the ultrasound energy is transmitted to fluids surrounding the distal portion 212. In addition, the housing 218 is rotatably coupled to a more proximal part of the distal portion 212 at a pin 219.

The housing 218 is coupled to two control wires 221 which extend through the shaft 206 to an actuator such as the actuator of a handle such as the handle 104 of FIG. 1 (e.g., the second actuator 128). Thus, by rotating the second actuator 128, one of the control wires 221 is pulled proximally while the other is pushed distally to rotate the housing 218 about the pin 219. This, in turn rotates the transducer 216 e.g., in a plane including the longitudinal axis L of the shaft 206 to generate a field of view delimited by the sweep of proximal and distal ends of the transducer 216 as shown in FIG. 5. Furthermore, as seen in FIG. 7, the pin 219 and the housing 218 are located distally at the port 210 and the elevator 232 of this embodiment. That is, the port 210 and the elevator 232 are located on the proximal part of the distal portion 212 that does not rotate when the housing 218 is rotated via the control wires 221. This permits the user to rotate the transducer 216 as desired to widen a field of view 223 provided to the user by the transducer 216 without moving a needle 209 or other device extended distally out of the port 210.

Similarly to the transducer 116, the transducer 216 may be rotated relative to the axis L through an angular range α which in this embodiment is between 15 and 180 degrees. The transducer 116 of this embodiment is coupled to a flexible conductor 224 that extends through the shaft 206 to the handle to power the transducer 216 and to convey data from the transducer 216 to a processor or computer as would be understood by those skilled in the art. As would be understood by those skilled in the art, the device 202 may be operated in the same manner described above for the device 102 except for the moving of the transducer 216 although this would be similar.

The shaft 206 may be mounted to a handle constructed substantially similarly to the handle 104 includes a first actuator used in a conventional manner to steer the device 202 (e.g., by bending the distal portion 212 in a desired manner) and a second actuator coupled to the control wires 221 so that rotation of the second actuator pulls one of the control wires proximally while the other control wire 221 is pushed distally to rotate the housing 218 relative to the more proximal portions of the device 202 thereby rotating the transducer 216 relative to the needle 209 and the target tissue site while keeping the needle 209 stationary.

Similarly to the device 102, the device 202 includes a sensor 222 is mounted at the interface between the housing 218 and the more proximal part of the distal portion 212 to detect the actual angular position of the housing 218 and the transducer 216. Those skilled in the art will understand that the sensor 222 may be, for example, an optical or magnetic linear encoder placed on the transducer itself or a rotary encoder on the rotating portion of the shaft. The sensor 222 of this embodiment is coupled to the proximal portion (e.g., the handle 104) via a conductor so that this information is transmitted to a control device (e.g., a processor or computer (not shown)) that receives information from the transducer 216 to construct the 3-dimensional map in the same manner described above. As indicated above, the steps for use of the device 202 are substantially the same as those described above for the device 102.

FIGS. 7 and 8 show a distal portion 312 of a device 300 of a further embodiment in which a transducer 316 is translated laterally within a housing 318 including a window 320 (e.g., along a path transverse to the axis L) via a gear mechanism 321 and a drive shaft 324 coupled for example to an actuator similar to the second actuator 128 of the handle 104. In the device 300, the drive shaft 324 is constructed similarly to the shaft 124 so that rotation of the actuator rotates the drive shaft 324. The distal end of the drive shaft 324 is coupled to a pinion 326 which is fixed in position relative to the housing 318. The pinion meshes with a rack 328 so that rotation of the drive shaft 324 rotates the pinion 326 which, in turn, moves the rack 328 laterally within the housing 318. The rack 328 is coupled to the transducer 316 so that movement of the rack 328 laterally moves the transducer laterally back and forth within the housing 318 through a range of, for example, 1 mm to 10 m to broaden the field of view provided to the user in a manner substantially similar to the previous embodiments.

It will be appreciated by those skilled in the art that changes may be made to the embodiments described above without departing from the inventive concept thereof. It should further be appreciated that structural features and methods associated with one of the embodiments can be incorporated into other embodiments. It is understood, therefore, that this invention is not limited to the particular embodiment disclosed, but rather modifications are also covered within the scope of the present invention as defined by the appended claims.

1-15. (canceled)

16. A flexible endoscope, comprising:
a flexible shaft extending from a proximal end connected to a handle to a distal portion configured to be inserted into a living body to a site adjacent to target tissue to be treated, the shaft including adjacent to a distal end thereof a port from which a tissue treatment device may be extended out of the shaft along a path; and
an ultrasound transducer within a housing coupled to a portion of the shaft extending distally of the port, the transducer being aimed so that the path passes through a field of view provided by the transducer to a user of the device, the transducer being movable relative to the port to broaden the field of view.

17. The endoscope of claim 16, wherein the transducer is movable laterally within the housing with respect to a longitudinal axis of the housing.

18. The endoscope of claim 16, wherein the transducer is rotatable within the housing about to a longitudinal axis of the housing.

19. The endoscope of claim 16, wherein the transducer is rotatable within the housing about an axis perpendicular to a longitudinal axis of the housing.

20. The endoscope of claim 17, wherein the transducer is coupled to a proximal actuator via a rotatable drive shaft and wherein the rotatable drive shaft is coupled to the transducer via a rack and pinion mechanism.

21. The endoscope of claim 18, wherein the port is configured as a lateral port aimed so that the device extended out of the port is aimed away from the longitudinal axis of the housing.

22. The endoscope of claim 21, wherein the lateral port is configured to aim a needle for fine needle aspiration along a path extending laterally away from the flexible endoscope.

23. The endoscope of claim 22, wherein the path is centered within the field of view of the transducer.

24. The endoscope of claim 16, wherein the housing is configured so that, when the transducer is moved relative to the port, the port remains substantially stationary.

25. The endoscope of claim 16, further comprising:
a sensor in the housing configured to provide to a control unit data corresponding to a current position so that images of the field of view may be accurately rendered.

26. The endoscope of claim 25, wherein the control unit produces a 3-dimensional map of the field of view.

27. The endoscope of claim 20, wherein the rotatable drive shaft is one of a solid wire, a tube, a coil, and a braid of medical grade metal.

28. The endoscope of claim 27, wherein the medical grade metal is one of nitinol and stainless steel.

29. The endoscope of claim 27, wherein the shaft is covered in a coating of one of PTFE and silicone lubricant.

30. A flexible insertion device, comprising:
a handle;
a flexible shaft extending along a longitudinal axis distally from the handle to a distal portion configured to be inserted into a living body to a site adjacent to target tissue to be treated, the distal portion of the shaft including a port in a lateral surface thereof from which a tissue treatment device may be extended out of the shaft along a path extending away from the longitudinal axis; and
a housing extending distally beyond the port, the housing including an ultrasound transducer therein, the transducer being aimed so that the path passes through a field of view provided by the transducer to a user of the device, the transducer being movable within the housing to broaden the field of view.

31. A method for treating tissue, comprising:
inserting into a living body a flexible endoscope, the endoscope including a flexible shaft which extends from a proximal end connected to a handle to a distal portion configured to be inserted into the living body to a site adjacent to target tissue to be treated;
extending out of the distal portion a tissue treatment device via a port adjacent to a distal end of the shaft; and
moving within a housing of the endoscope distal to the port, an ultrasound transducer of the endoscope so that a field of view generated by the transducer includes a path along which the device extends.

32. The method of claim 31, wherein the transducer is movable laterally within the housing with respect to a longitudinal axis of the housing.

33. The method of claim 31, wherein the transducer is rotatable within the housing about to a longitudinal axis of the housing.

34. The method of claim 31, wherein the transducer is rotatable within the housing about an axis perpendicular to a longitudinal axis of the housing.

35. The method of claim 32, wherein the transducer is coupled to a proximal actuator via a rotatable drive shaft and wherein the rotatable drive shaft is coupled to the transducer via a rack and pinion mechanism.
